(19) United States
(12) Patent Application Publication
(10) Pub. No.: US 2025/0084459 A1
(43) Pub. Date: Mar. 13, 2025

(54) Expanding the Molecular Processing and Biosensing Capabilities of a Single-Construct Quantum Dot-Based Biosensor By Selectively Controlling Energy Transfer Pathways

(71) Applicant: The Government of the United States of America, as represented by the Secretary of the Navy, Arlington, VA (US)

(72) Inventors: Igor L Medintz, Washington, DC (US); Matthew Chiriboga, Washington, DC (US); Shelby L. Hooe, Washington, DC (US); David A. Hastman, Washington, DC (US); Niko Hildebrandt, Ontario (CA); Sebastian A. Diaz, Washington, DC (US); Kimihiro Susumu, Washington, DC (US)

(73) Assignee: The Government of the United States of America, as represented by the Secretary of the Navy, Arlington, VA (US)

(21) Appl. No.: 18/776,176

(22) Filed: Jul. 17, 2024

Related U.S. Application Data
(60) Provisional application No. 63/537,855, filed on Sep. 12, 2023.

(51) Int. Cl. C12Q 1/6818; C12Q 1/37

(52) U.S. Cl. CPC C12Q 1/6818; C12Q 1/37

(57) ABSTRACT

An energy transfer platform involves a CdSe/ZnS core-shell quantum dot (QD) scaffold having four additional components: terbium metal chelate, ruthenium(II)-phenanthroline, AlexaFluor 647, and Cy5.5. These are positioned in a self-assembled fashion on the QD surface through the utilization of peptide-PNA and DNA bioconjugate linkers. The modular platform can operate to sense multiple different events or targets simultaneously, including sensing of genetic moieties and enzymatic targets.

### CROSS-REFERENCE TO RELATED APPLICATIONS

This application is a non-provisional of, and claims priority to and the benefits of, U.S. Provisional Patent Application No. 63/537,855 filed on Sep. 12, 2023, the entirety of which is herein incorporated by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211680.

### BACKGROUND

Semiconductor quantum dot nanocrystals (QDs) possess remarkable optical, electronic, and physical properties. Furthermore, they offer diverse possibilities for the construction of intricate energy transfer pathways: QDs exhibit efficient Förster resonance energy transfer (FRET) capabilities, enabling them to serve as efficient energy donors or acceptors for various optical materials, such as organic fluorophores, metal chelates, and metallic nanoparticles. Moreover, QDs can effectively transfer energy to organometallic ruthenium complexes via photoinduced electron transfer (PET). Through the strategic utilization of bioconjugate linkers, including nucleic acid and peptide derivatives, each of these optical material types can be precisely positioned and self-assembled on the surface of QDs. The inherent modular design of these systems, coupled with the flexibility of measurement parameters, unlocks an extensive array of distinctive output spectral signatures. This notable feature holds immense value for a wide range of applications, encompassing biosensing, molecular computing, and encryption.

A need exists for new QD-based sensing technology.

### BRIEF SUMMARY

In a first embodiment, a modular energy transfer platform includes a CdSe/ZnS core-shell quantum dot (QD) scaffold having four additional components bound thereto, the additional components comprising terbium, ruthenium, AlexaFluor 647, and Cy5.5, wherein each of the additional components are bound to the QD scaffold via an arm, each arm comprising a peptide-PNA strand comprising (1) a peptide nucleic acid (PNA) comprising a PNA sequence and (2) a peptide comprising a polyhistidine sequence effective to bind the peptide-PNA strand to the QD; and the arm comprising ruthenium features the ruthenium conjugated to the PNA sequence via a modified peptide derivative.

Another embodiment is a method of preparing a modular energy transfer platform involving heating a mixture of anchor, template, and probe strands under conditions sufficient to denature DNA found within, and allowing them to assemble into arms, and then without further purification, allowing these arms to bind to a quantum dot to form the modular energy platform of the first embodiment.

A further embodiment is a method of analysis comprising contacting a modular energy transfer platform of the first embodiment with a sample comprising (1) nucleic acids operable to displace the terbium, AlexaFluor 647, and/or Cy5.5 from the scaffold; and/or (2) a protease operable to separate the ruthenium from the scaffold; and measuring a change in the optical properties of the scaffold as a result of the contacting.

### BRIEF DESCRIPTION OF THE DRAWINGS

FIG. 1A offers a schematic representation of the single-construct quantum dot (QD)-based biosensor. Stars depict the optical components of Tb, Ru, AF, Cy, while the QD is shown as the central crystal. The bioconjugate arm structure, composed of a peptide-PNA strand terminated by a (His)6, links each fluorescent component to the QD. The bioconjugate arm anneals to a partially complementary DNA template strand, which captures the probe strand labeled with one of the optical components. The Ru component is linked to the QD through a modified peptide linker. Inputs such as “sensing DNA” and “sensing enzyme” can be added in excess post-assembly to catalyze the dissociation of each optical component. FIG. 1B is a simplified illustration of self-assembly of the bioconjugate arm by mixing the strands in a 1:1 ratio. After heating to 95° C. for 45 minutes (for example) and allowing the mixture to cool, the DNA strands anneal, enabling utilization without a need for further purification.

FIG. 2A presents the normalized absorbance and fluorescence emission spectra of each component: Tb, QD, Ru, AF, and Cy. It is important to note that Ru exhibits no observable fluorescence and does not require spectral overlap for transfer, but is included in the figure as it is capable of absorbing photons in the 400-600 nm range. These spectra, in conjunction with the photophysical constants listed in Table 1 (found in FIG. 9), were utilized to calculate the J-overlap integral shown in FIG. 2B for the transfer steps: Tb to QD, QD to AF, QD to Cy, and AF to Cy.

FIG. 3 provides a schematic representation of the experimental setup. The results of each experiment depend on three sets of parameters. First the excitation parameters govern the behavior of the light source utilized to probe the state of the biosensor. Second, the assembly parameters determine the behavior of the biosensor itself, including the number of available states and the unique spectral readout it produces. Third, the detector parameters dictate the process of generating the fluorescence emission spectral readout. The figure illustrates example parameters and includes a visual map demonstrating how energy is transferred throughout the system. It also highlights the contribution of each fluorescence readout to the observed emission, represented as a linear combination.

FIGS. 4A-4D illustrate the interactions between the optical components, leading to changes in the observed spectrum, with two scenarios: a 300 μs lag time (A) and no lag time (B). The process begins with the presence of only the QD (black curve), and as each component is added to the system, a new fluorescence channel is introduced or the emission of existing components is modified, until all components are present. By utilizing the individual spectra of each component (FIG. 2), it becomes possible to estimate the approximate emission of each component. In FIGS. 4C and 4D, the estimated emissions of the Tb, QD, AF, and Cy components are shown for the full system (black dashed) measured with a 300 μs lag time (C) and no lag time (D).

FIGS. 5A-5D demonstrate how the energy transfer properties of the biosensor can be optimized and customized by controlling the relative proportions of each optical component. Since the QD serves as the central scaffold, the figure showcases the impact of titrating increasing amounts of (A) Tb, (B) Ru, (C) AF, and (D) Cy in relation to the QD on the fluorescence emission from the sensor. Panel A was measured with a 300 μs lag time, while panels B to D were measured with no lag time. The emission spectra in FIGS. 5A and 5B have been normalized for clarity. Inset panels provide visual representations of the change in integrated emission based on the ratio of each component to the QD, with the emission of QD alone set to zero a.u.

FIG. 6 presents a schematic representation of the data processing workflow employed to obtain a processed emission dataset. Initially, for a given sample characterized by specific excitation, assembly, and detection parameters, there are n=3 independent measurement repeats. Consequently, the total number of measurements equals 3n, accounting for all n distinct samples. Each of these 3n spectra undergoes individual spectral unmixing into fluorescence channels, achieved by solving a linear least squares problem through singular value decomposition (SVD) and minimizing the L2-norm of the residuals. Once spectrally unmixed, the repeats are processed by calculating the mean and sample standard deviation. Simultaneously, the raw spectra are processed by computing the mean, sample standard deviation, and applying smoothing if necessary.

FIGS. 7A-7D show input titration curves illustrating the variation in fluorescence signal intensity as a response to the dissociation of optical components. The fluorescence channels of Tb (A), QD (B), AF (C), and Cy (D) can be intentionally optimized or fine-tuned by adjusting the concentration of excess Tb, Ru, AF, or Cy input in solution. Each point on the scatterplot corresponds to the change in integrated emission for a specific fluorescence channel, considering a particular ratio of input to label and an initial value of zero a.u. Exponential growth/decay functions (solids) were fitted to the data, exemplifying the alteration of emission in response to increasing input. All panels demonstrate the changes in response within a fully assembled system comprising Tb, QD, Ru, AF, and Cy. Measurements for FIGS. 7A and 7B used a 300 μs lag time, while those in FIGS. 7C and 7D were obtained without a measurement lag time. It is important to note that the Tb, AF, and Cy inputs are DNA strands, whereas the Ru input is enzymatic in nature.

FIGS. 8A-8F present schematics and example spectra of a two-input molecular logic gate. The gate operates using a simplified version of the sensor, consisting only of the QD, Ru, and AF components. As a result, there are only two fluorescence channels present: QD and AF. In this system, only the inputs Rui and AFi will affect the behavior. A through D illustrates the scenarios where (A) neither, (B) the Rui only, (C) the AFi only, or (D) both Rui and AFi are present. Dotted lines indicate arbitrary threshold values that correspond to the behavior of an AND gate (black) as well as an OR gate (red). FIGS. 8E and 8F display the logical circuit diagram associated with each gate and provide the truth tables representing the Boolean computation performed by each gate. In FIGS. 8A-8D the full spectrum is also presented (dashed black) for clarity, accompanying the unmixed spectra.

FIG. 9 presents Table 1 showing selected photophysical constants associated with each optical component. The values of R₀ assume random dynamic dipole averaging (κ²=⅔). Values of quantum yield and ext. coeff. are manufacturer reported.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

Described herein is a multi-component energy transfer platform constructed on a single QD scaffold. This design follows a modular approach, allowing for the inclusion or exclusion of each component in the assembly based on specific requirements. The fully configured system comprises five distinct (nano)materials: Lumi4 terbium metal chelate (Tb), CdSe/ZnS core-shell QDs, ruthenium(II)-phenanthroline (Ru), AlexaFluor 647 (AF), and Cy5.5 (Cy). Each optical material contributes to the overall performance of the sensor and offers unique advantages.

The incorporation of Tb allows exploitation of their long excited state lifetime, which is a distinctive characteristic of this moiety. Leveraging the Tb donor as an initial sensitizer allows for time-gated measurements and spectral tuning based on lag times. QDs, known for their highly efficient photonic relay capabilities, act as the central scaffold and engage in energy transfer with the other materials, facilitating the efficient flow of energy within the system and subsequent signal transduction. The inclusion of a Ru motif introduces a distinct energy transfer mechanism through PET, enabling the quenching of QD photoluminescence. This mechanism complements the traditional FRET steps and provides precise control over signal amplitude by quenching QD-excited states. Furthermore, the incorporation of AF and Cy enables an energy transfer process with an overlapping configuration, creating a concentric FRET circuit. This configuration offers two significant advantages: it allows for signal amplification of Cy by improving energy transfer efficiency, and enables the generation of additional output signals. Concentric FRET circuits involve multiple fluorophores which compete for or alternatively control energy transfer. This leads to augmented control over the system by manipulating inputs and managing competition between them.

This approach employs a bioconjugation strategy involving peptide nucleic acid (PNA), a unique nucleic acid derivative where the nucleobases are connected by a peptidyl backbone instead of the traditional phosphate backbone. This distinctive material offers significant advantages, as it allows for the inclusion of a terminal polyhistidine to the peptide backbone while preserving the nucleotide hybridization capability in the PNA region. This enables efficient attachment of optical components to the QD scaffold while maintaining molecular recognition properties. A DNA template anneals to the PNA and facilitates the capture of a dye-labeled probe strand. Such a hybrid peptide-PNA and DNA template approach not only allows for rapid prototyping through the system's modularity, enabling quick swapping of inputs by modifying DNA recognition sequences, but it also adds a dynamic aspect to the system's behavior. By leveraging toehold strand displacement, the system's output can be modified post-assembly, providing an additional layer of control and adaptability. This bioconjugation strategy offers enhanced flexibility and adaptability, facilitating versatile customization and dynamic control of its behavior. Moreover, the inherent properties of nucleic acid hybridization make this system easily extendable to RNA and other genetic materials, broadening its potential applications. Finally, the system can be modified to utilize traditional peptide bioconjugation, providing a more straightforward approach for linking optical components to the QD scaffold. This opens up opportunities to incorporate enzymatic recognition sequences into the peptides, enabling the sensing of protease activity and expanding the sensing capabilities of the system. Overall, this bioconjugation strategy combines the benefits of PNA, DNA templates, and traditional peptides, allowing for efficient assembly, dynamic control, and diverse functionality.

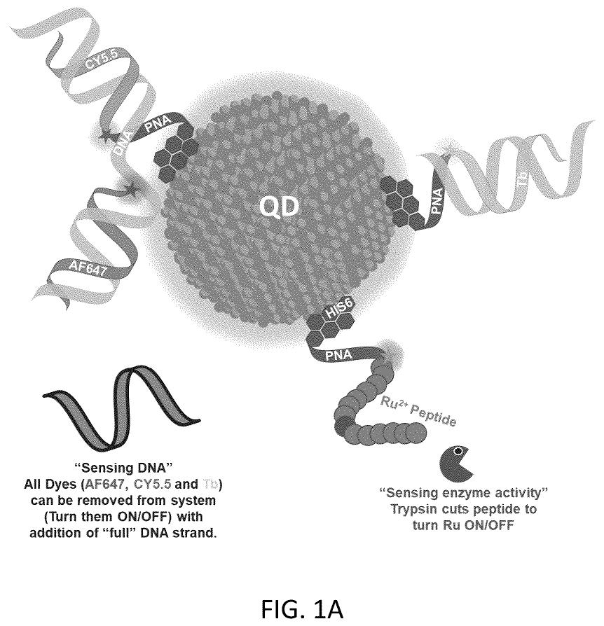

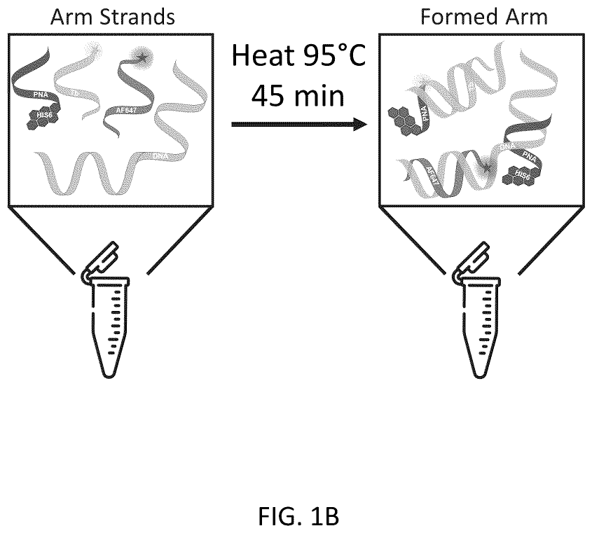

The energy transfer platform as described herein involves a QD scaffold having four additional components: Tb, Ru, AF, and Cy positioned in a self-assembled fashion on the QD surface through the utilization of peptide-PNA and DNA bioconjugate linkers (FIG. 1A). Each component plays a role in the overall performance of the sensor, contributing specific advantages and functionalities.

Furthermore, each component is designed to self-assemble under controlled conditions.

In the complete system, each fluorescent material assembles itself onto the surface of the QD using the peptide-PNA/DNA template linker, termed an “arm” (FIG. 1B). The arm includes up to three sub-components: a peptide-PNA anchor (brown in FIGS. 1A and 1B), a DNA template guide (gray in FIGS. 1A and 1B), and an optional probe strand (colored in FIGS. 1A and 1B) covalently connected to the optically active label (colored star in FIG. 1A). The peptide-PNA anchor, terminated by a (His)6-motif (brown hexagon in FIG. 1A), enables attachment to the QD surface. The PNA region of the peptide-PNA anchor complements a subsequence of the DNA template, promoting hybridization while leaving an overhang available for capturing the probe strand. The probe strand, modified with the desired fluorophore, is designed to be complementary to the available overhang, which guides the probe into position for energy transfer. To assemble the arm, the necessary strands (anchor, template, and probe) are mixed in a 1:1:1 ratio and heated to 95° C. for 45 minutes (FIG. 1B). The arm can then be used without further purification. Finally, the Ru component is directly conjugated to the QD using a modified peptide derivative, where a (His)6 sequence and Ru component are located at opposite termini. Further details on the components and their assembly can be found in the Appendices submitted herewith.

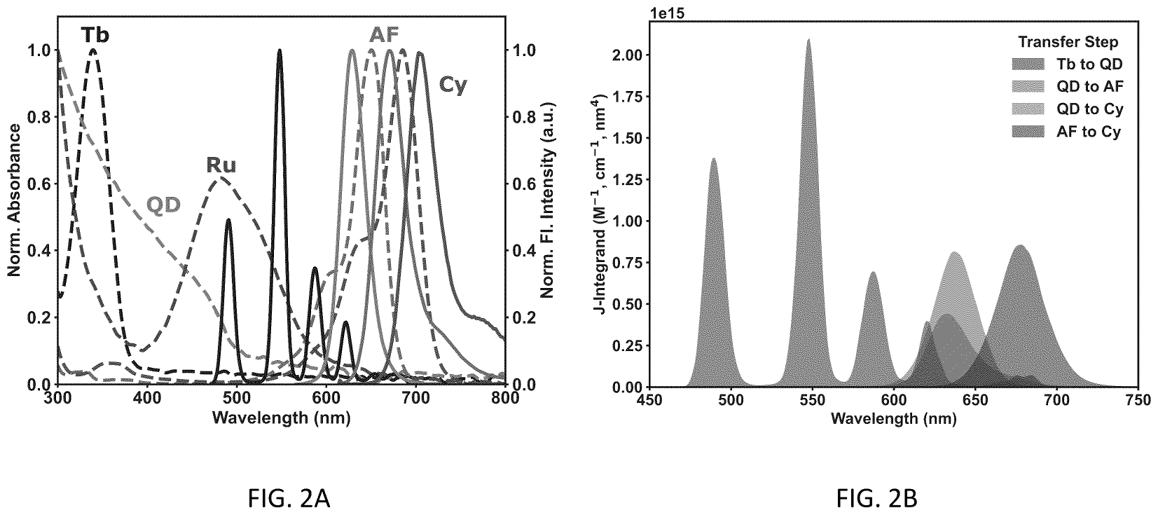

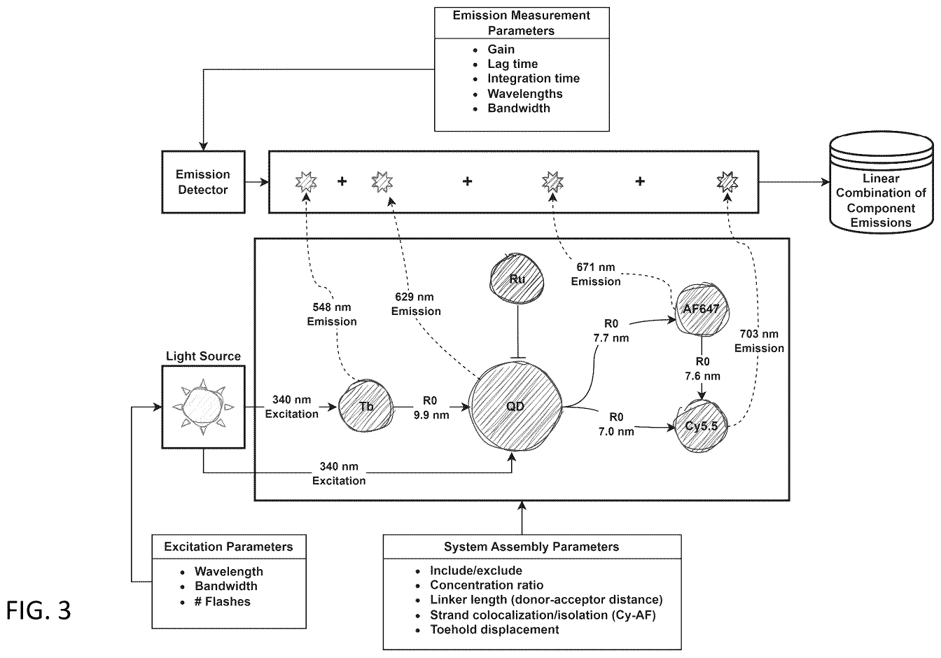

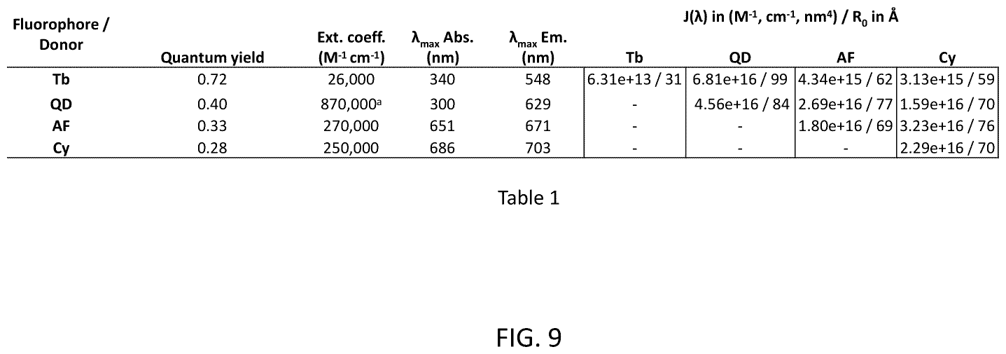

The spectral components of the system are designed such that the energy levels of each component enable a sequential energy transfer cascade. This fact is conveyed through the absorbance and emission spectra of the components (FIG. 2A) and the corresponding spectral overlap (FIG. 2B). These features are quantified in Table 1 (FIG. 9) along with estimated Förster radii assuming random dynamic dipole averaging (κ²=⅔). Once coupled to an external light source, the flow of energy is outlined by arrows in FIG. 3. For example, assuming a 340 nm excitation, the Tb will transfer energy to the QD. The QD can transfer energy to both AF and Cy, while also capable of being quenched by PET from the Ru. Finally, the AF can transfer energy to the Cy which acts as a final collection point.

Depending on the parameters of the system, a relative proportion of each fluorescent component will emit photons with emission maxima at 548 nm, 629 nm, 671 nm, and 703 nm for Tb, QD, AF, and Cy respectively (arrows with dashed lines in FIG. 3). The fluorescent emission is then recorded by the detector as a linear combination of the component emissions. From this point, the data undergoes subsequent processing and spectral unmixing in preparation for analysis (see Appendices for additional detail).

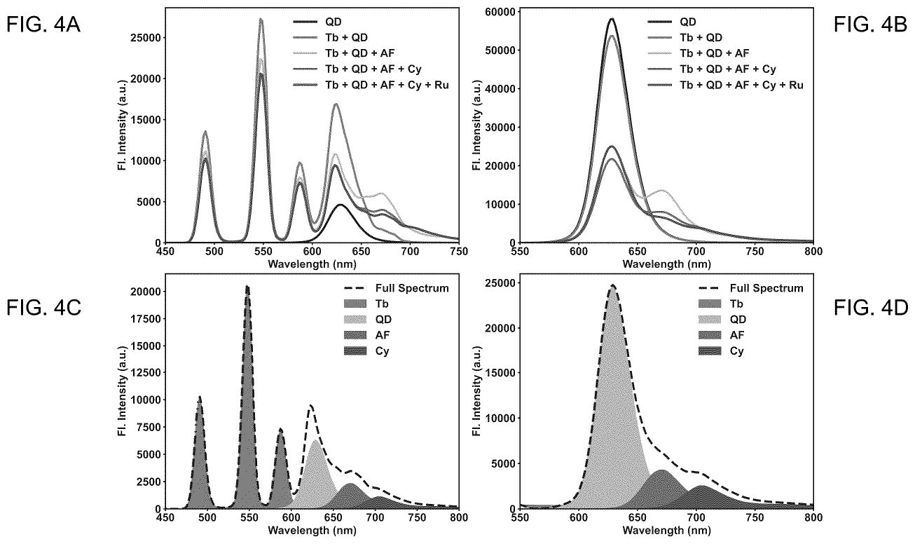

One particular advantage of this system is that employing Tb as an initial sensitizer enables time-gated measurements. The UV absorbance spectra of Tb and QD exhibit a significant overlap, as illustrated in FIG. 2A. Consequently, an optical excitation of 340 nm directly stimulates both Tb and QD. However, a disparity exists between the lifetimes of Tb (on the order of 2×10⁻³ s) and QD (on the order of 10×10⁻⁹ s). Leveraging a time-gated detection window effectively enhances multiplexing capabilities, allowing for the differentiation between energy transfer from Tb and direct excitation of QD. Specifically, FIG. 4A showcases the fluorescence emission spectra with a 300 μs detection lag time, enabling the direct excitation of QD to dissipate, thereby isolating the energy transfer from Th. While in contrast, FIG. 4B portrays the same samples measured without a lag time between excitation and detection. In this scenario, direct excitation of QD and its subsequent emission significantly surpasses that of Tb, demonstrating the superiority of time-gated detection in suppressing unwanted signals. Furthermore, FIGS. 4A-4C provide insight into the intricate process of energy transfer when different optical components are incorporated, highlighting its impact on the observed spectra. The observed spectrum is formed through a linear combination of individual components, enabling the extraction of contributions from each component by solving a set of linear equations. By examining the fully assembled system (FIGS. 4A and 4B), this process can be observed in FIGS. 4C and 4D. Ultimately, this analytical approach allows for a comprehensive analysis of each construct in terms of fluorescence channels, providing valuable insights into both the overall system state and the state of each individual fluorescent component.

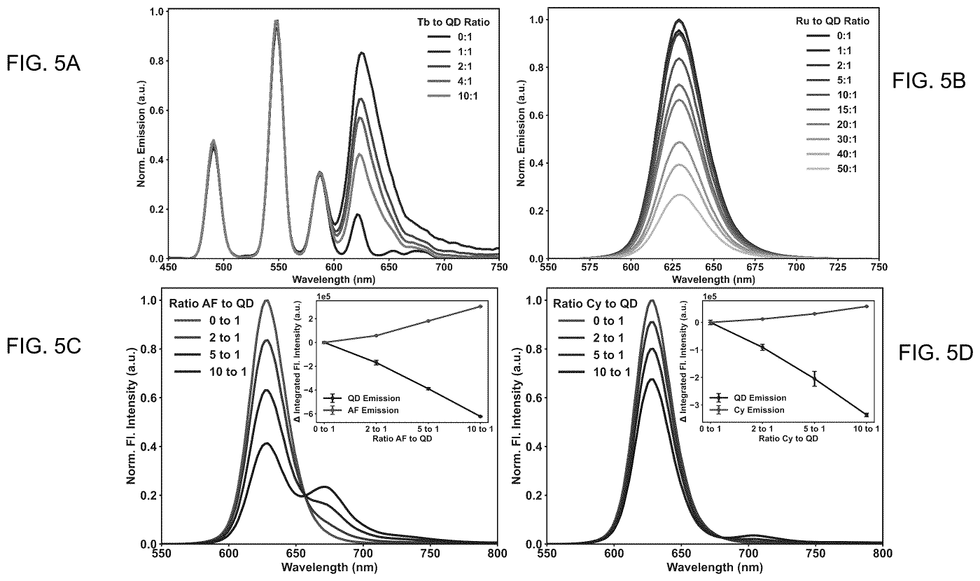

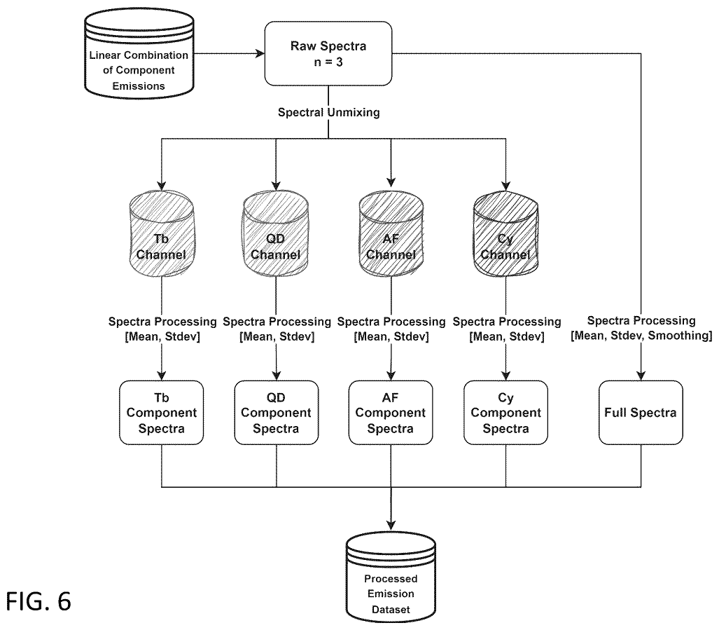

In addition to the binary inclusion or exclusion of components, precise control over the spectral output of the system can be achieved by optimizing the relative amounts of each component. To illustrate this, FIG. 5A demonstrates the emission changes resulting from titrating increasing amounts of Tb relative to QD. Given the excess of donor relative to acceptor, the energy transfer efficiency is unlikely to increase significantly. Instead, this ratio allows for optimization of signal gain. In FIG. 5B, increasing amounts of Ru-modified peptide are mixed with QD, resulting in an increasing quenching effect due to PET. The impact of increasing AF and Cy titrations relative to QD can be observed in FIGS. 5C and 5D, respectively. Since the observed fluorescence data is a linear combination of the contributions from each optical component, the data can be processed to obtain the emission of each individual component. This data processing workflow is outlined in FIG. 6, where a linear system of equations is solved to express each raw spectrum in terms of fluorescence channels as well as the full spectra. The scatterplot insets in FIGS. 5A-5D represent the change in the integrated emission intensity of the corresponding fluorescence emission channels obtained through the process outlined in FIG. 6.

The properties of DNA and peptides provide a physical mechanism for both signal inhibition and transduction. By leveraging toehold displacement reaction chemistry, one can dynamically control the relative abundance of each DNA optical component in solution post-assembly. This forms the basis for the DNA-sensing component. Target acts as the displacing strand. To selectively remove a specific optical probe strand post-assembly, an excess amount of sequence identical input DNA (FIG. 1A) is introduced. This introduction of excess input strands shifts the chemical equilibrium to counteract the change, resulting in the displacement of the optical probe strands by the unlabeled input strands. This process preserves the integrity of the arm structure, rendering it theoretically reversible given the introduction of additional optical probes. However, the Ru input is an exception to this reversibility as it cannot be removed through toehold displacement due to its attachment via a modified peptide. In this case, the input is a suitable protease enzyme, for example trypsin, (FIG. 1A) that cleaves the peptide and releases the Ru. The enzymatic cleavage of the peptide is a covalent process and therefore irreversible, unlike the toehold displacement reactions. The combination of these four processes establish a single sensor can simultaneously detect three different target DNA strands along with the activity of an enzyme—a protease in this example.

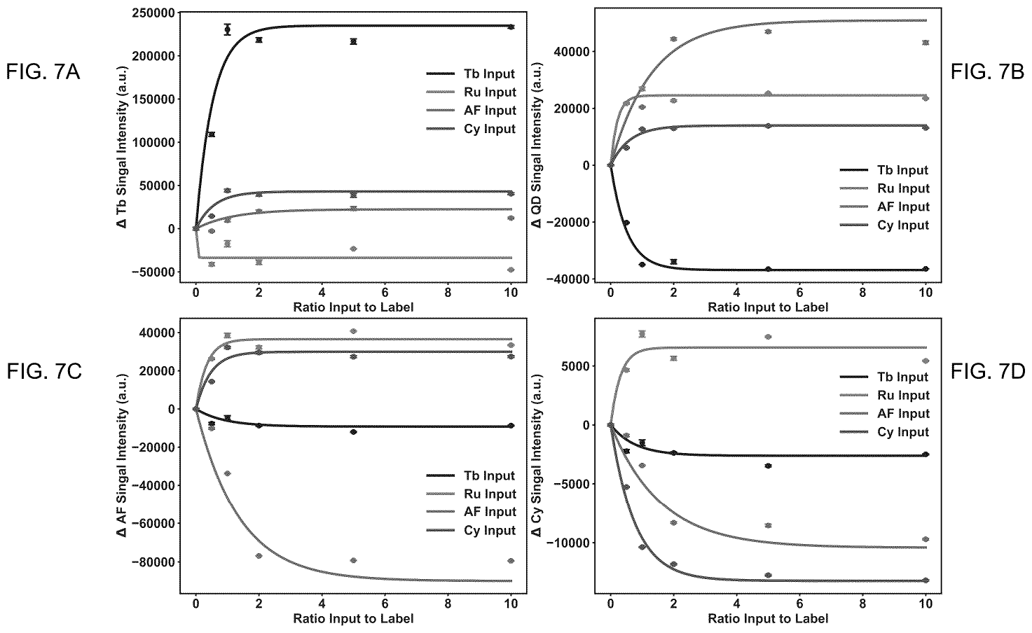

The impact of the introduced input strands on the system can be observed in FIGS. 7A-7D. Each panel represents examples of monitoring the change in integrated emission intensity for a specific fluorescence channel when an input is added in excess. To simplify, the emission channel of each optical component is isolated from the full spectrum to observe how the emission of that component changes upon the introduction of each input. FIG. 7A provides an example of how the Tb channel signal can be adjusted in response to the given inputs. For this panel, we utilize a 300 μs lag time and a 1,500 ms integration window, as the Tb signal is most effectively measured after a delay. As anticipated, the introduction of the Tb input (black) leads to a significant increase in the Tb channel signal, since the Tb probe dissociates from the QD, thus it is no longer being quenched. Furthermore, we observe a weak increases in the Tb signal with the introduction of the AF and the Cy inputs. This observation aligns with the spectral overlap between Tb and these dyes, and the potential for some quenching due to energy transfer. Similarly, FIG. 7B illustrates the signal from the QD channel. To effectively demonstrate the effects of the Tb input on the QD, this panel also employs the same lag time and integration window as before. Since the QD serves as the central scaffold and interacts with each of the other optical components, we anticipate signal changes with each input. The introduction of the Tb input (black) leads to a decrease in the QD signal, as there is no longer energy transfer from Th. Conversely, the Ru input causes an increase in the QD signal due to the removal of the PET pathway. Lastly, the AF and Cy both exhibit increases in the QD signal because of the elimination of FRET quenching. FIGS. 7C and 7D depict the changes in signal for the AF and Cy channels, respectively, without any lag time delay. Consequently, both panels show minimal alterations in signal intensity in response to the Tb input (black), as the energy transfer primarily originates from direct excitation of the QD. Conversely, both channels exhibit an increase in signal when the Ru input is introduced. This is because both AF and Cy are downstream of the QD energy transfer and are subject to quenching caused by Ru-induced PET. Moving on to the AF channel in FIG. 7C, the AF input leads to a strong decrease in the AF signal due to the absence of sensitization from the QD. Conversely, the Cy input results in a strong increase of AF signal, as there is no longer quenching from Cy. Finally, for the Cy channel in FIG. 7D, the signal decreases in response to both the AF input and the Cy input, with the Cy input change being stronger. This can be attributed to the concentric FRET, and the removal of AF inhibiting the sensitization of Cy. However, in this case, the QD is still capable of transferring some energy to Cy, resulting in a weaker signal decrease compared to the scenario where Cy is directly removed, leading to the loss of both QD and AF sensitization pathways.

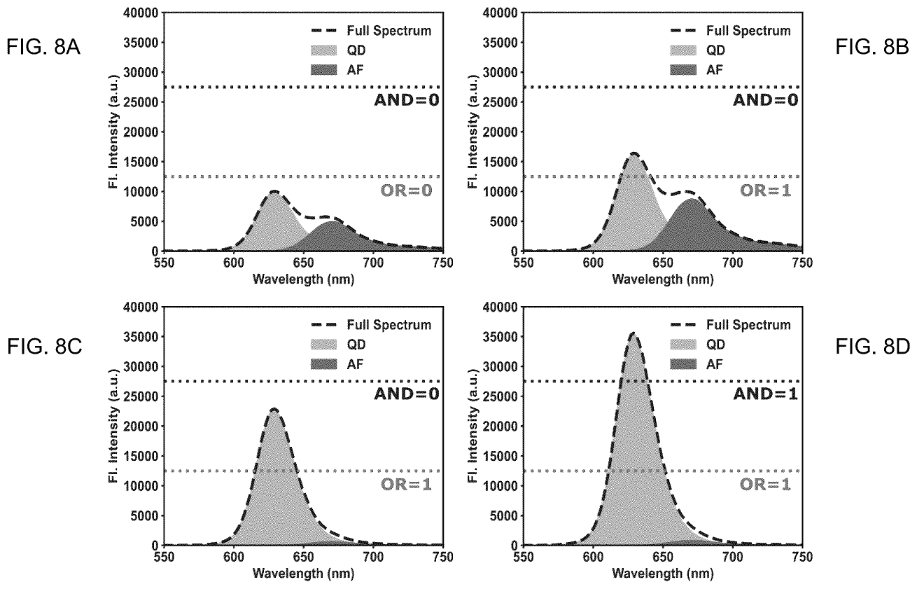

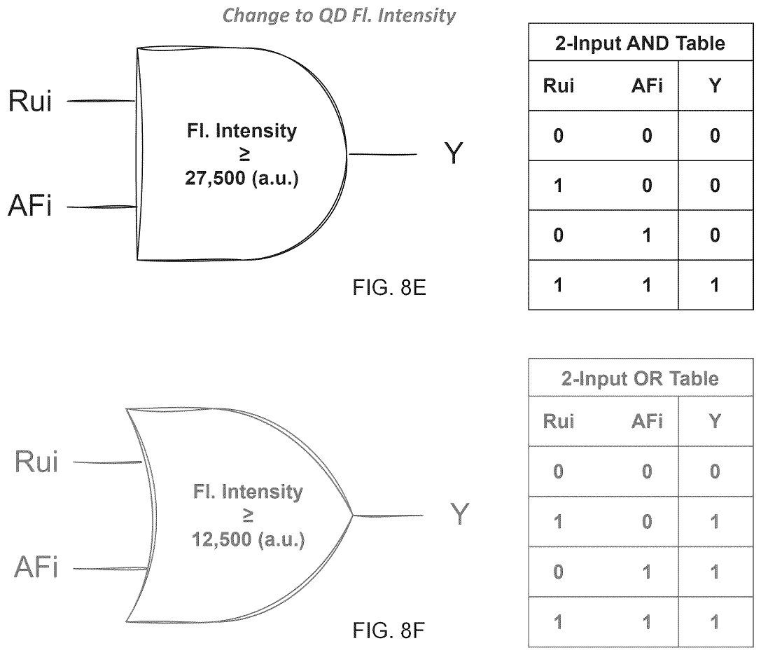

The combination of fluorescence channels and input strands can also allow the assembly to act as an information processing device. This construct enables the design of excitonic molecular logic gates, serving as a physical analogy to the building blocks of digital circuits. To illustrate this concept, let's consider a simplified configuration of the sensor, incorporating QD, Ru, and AF. The Ru component quenches the QD through PET, while the AF quenches the QD via FRET. This cooperative effect can be harnessed for sensing and Boolean operations. By utilizing the Ru input (Rui) and AF input (AFi), one can create basic two-input logic gates based on the changes in energy transfer and subsequent emission from the QD fluorescence channel, as depicted in FIGS. 8A-8F. There the full spectra (black dashed line) representing the overall system behavior are resolved into the QD and AF fluorescence channels. One can systematically introduce each input in excess to selectively remove specific components from the system. For instance, FIG. 8A displays the spectra when neither Rui nor AFi is present. Subsequently, FIGS. 8B and 8C demonstrate the spectral changes upon introducing Rui and AFi, respectively. As expected, in both scenarios, the QD signal increases due to the decrease in quenching. Finally, in FIG. 8D, both Rui and AFi are simultaneously introduced, resulting in the most substantial increase in QD signal as both quenching pathways are eliminated. In this scenario, by defining an appropriate switching threshold, one can construct both an AND gate and an OR gate. For instance, setting the switching threshold to a QD maximum intensity greater than 27,500 arbitrary units (a.u.) (FIG. 8E) achieves the AND gate functionality. Alternatively, when the threshold is set to a QD maximum intensity greater than 12,500 a.u. (FIG. 8F), an OR gate is achieved. While these examples are simplistic, utilizing only a fraction of the complete system, they serve as proof of concept. The system's remarkable modularity, the unique properties of the optical components, and the versatility of data processing techniques allow for the extension of this system to much more complex sensing and logic operations. With its inherent flexibility and the potential for sophisticated expansion, this system holds significant promise for a wide range of applications in sensing and advanced logic operations.

### Advantages

The modular energy transfer platform described herein combines biosensing and molecular logic operations. The fully configured system incorporates three variations of photonic energy transfer: time-gated FRET, PET, and concentric FRET. The integration of these three distinct mechanisms of energy transfer into a single molecular unit offers several unique advantages not believed to have been previously achieved. Collectively, this system provides the following benefits:

(1) Rapid prototyping: The ability to swap out different arm strands allows for easy inclusion or modification of desired optical components, facilitating rapid prototyping and system customization.

(2) Modular sensing of genetic moieties: The modularity of the template strand enables the sensing and detection of DNA, RNA, and other genetic entities, expanding the configuration of the system.

(3) Enhanced energy transfer control: By utilizing long-lifetime Tb and eliminating emission from short-lifetime species, the system effectively isolates energy transfer and provides precise control over the process.

(4) The potential intensity of fluorescence in each channel or even multiple channels simultaneously can be controlled by the ratios of relative donors to acceptors assembled on the QD; this also provided for control over relative FRET efficiency.

(5) The ability to sense multiple different events or targets simultaneously.

(6) The sensor is amenable to detecting many other proteases which may be targeted by either assembling Tb/dye-labeled or Ru-labeled peptides to the QD scaffold.

(7) The sensor is amenable to detecting other coupled enzymatic processes such as phosphorylation as coupled to a phosphorylation specific protease. Other types of enzymatic activity that modify the structure conformation of peptide, oligonucleotides or other substrates that bring a dye/Tb or Ru into similar proximity to the QD may also be feasible

(8) Time-gate tuning: Fine-tuning of the spectral output is achievable by modifying time-gate parameters such as lag time and integration window, allowing for customization and optimization.

(9) Non-fluorescent inhibitor control: The introduction of Ru and PET steps enables the use of non-fluorescent quenching to control the signal gain of specific outputs or serve as analog inhibitor switch, broadening the range of functional possibilities.

(10) The sensor is amenable to other biosensing modalities such as being attached to a surface or a paper-based disposable substrate. Sensor output is amenable to being monitored by cell-phone cameras

(11) The assembly design can be expanded and additional complexity added by incorporating more excitation wavelengths than demonstrated here and then looking at ratios of emission spectra or channels to each other.

(12) Compatibility with electrochemical detection: The coupling of Ru to other electrochemical detection methods expands the potential applications and synergies with complementary detection techniques.

(13) Optimized spectral interactions: The inclusion of AF and Cy concentric FRET, as opposed to disparate FRET steps, offers the ability to fine-tune the spectral output and optimize interactions within the system. However, the system can also allow for a more coarse-grained approach if desired.

(14) Dynamic control over energy transfer: Post-assembly, energy transfer can be dynamically controlled through the use of unlabeled complementary input strands, providing flexibility in system response and adaptability.

(15) Detection of protease activity: The modified peptide linker of Ru enables the detection of protease activity, expanding the system's capabilities into enzymatic sensing.

(16) Complex logic gate design: The system enables the design and realization of complex logic gates with up to five inputs, facilitating advanced computational operations within a molecular framework.

(17) Extensive parametric control: By combining various system assembly parameters, excitation parameters, emission measurement parameters, data processing parameters, analysis, and thresholding parameters, this system offers one of the largest known spaces of possible unique spectral signatures. This extensive control allows for fine coarse-grained optimization and customization of the system's behavior.

(18) The system can be assembled with other QDs that emit at different wavelengths and appropriate choices of FRET dye acceptors and donors along with different electron transfer acceptors.

(19) The use of peptides and DNA as interchangeable components, it is not fixed to Ru coupled only to peptide but can also be Ru-DNA which would sense a nuclease.

(20) The ratios of each component assembled to the central QD can be controlled during assembly to control the relative rate of FRET or electron transfer.

(21) The sensor is capable of detecting almost anything that is hydrolytic as long as the appropriate target molecule is inserted between the QD and the dye, Tb, or Ru. For example a small fatty acid could be cleaved by a lipase.

(22) The sensor is capable of detecting complex multistep biological process where, for example, a protease site on a peptide has to be phosphorylated or dephosphorylated for cleavage to happen, thus providing an indirect sensor for that activity or coupled activity.

In summary, the proposed energy transfer platform presents a multitude of advantages and new features, including modularity, genetic sensing capabilities, control over energy transfer, spectral tuning, inhibitor-based control, compatibility with complementary detection methods, optimized spectral interactions, dynamic control, protease activity detection, complex logic gate design, and extensive parametric control. These attributes demonstrate the substantial potential of this system for a wide range of applications in sensing, computing, and advanced molecular technologies not before achieved on a single molecular platform.

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Medintz, I. L. and H. Mattoussi. Quantum dot-based resonance energy transfer and its growing application in biology. Phys. Chem. Chem. Phys. 11, 17-45 (2009).
2. Medintz, I. L., Clapp, A. R., Mattoussi, H., Goldman, E. R., Fisher, B., Mauro, J. M. Self-assembled nanoscale biosensors based on quantum dot FRET donors. Nature Materials 2:630-638 (2003).
3. Algar, W. R., Wegner, D., Huston, A., Blanco-Canosa, J. B., Stewart, M. H., Armstrong, A., Dawson, P. E., Hildebrandt, N. and Medintz, I. L. Quantum Dots as Simultaneous Acceptors and Donors in Time-Gated Forster Resonance Energy Transfer Relays: Characterization and Biosensing. Journal of the American Chemical Society 134, 1876-1891 (2012).
4. Spillmann, C. M; Ancona, M. G.; Buckhout-White, S.; Algar, W. R.; Stewart, M. H.; Susumu, K.; Huston, A. L.; Goldman, E. R.; Medintz, I. L. Achieving Effective Terminal Exciton Delivery in Quantum Dot Antenna-Sensitized Multistep DNA Photonic Wires. ACS Nano 7, 7101-7118 (2013).
5. Algar, W. R.; Kim, H.; Medintz, I. L.; Hildebrandt, N. Emerging Non-Traditional Forster Resonance Energy Transfer Configurations with Semiconductor Quantum Dots: Investigations and Applications. Coordination Chemistry Reviews 263-264, 65-85 (2014).
6. Clapp, A. R., Medintz, I. L., Fisher, B. R., Uyeda, H. T., Goldman, E. R., Bawendi, M. G., Mattoussi, H. Quantum Dot-Based Multiplexed Fluorescence Resonance Energy Transfer. J. Am. Chem Soc. 127, 18212-18221 (2005).
7. Medintz, I. L., Farrell, D., Susumu, K., Trammell, S., Deschamps, J. R., Brunel, F., Dawson, P., and Mattoussi, H. Multiplex charge transfer interactions between quantum dots and peptide-bridged ruthenium complexes. Analytical Chemistry. 81, 4831-4839 (2009).
8. Algar, W. R Ancona, M. G.; Malanoski, A. P.; Susumu, K.; and Medintz, I. L. Assembly of a Concentric Forster Resonance Energy Transfer Relay on a Quantum Dot Scaffold: Characterization and Application to Multiplexed Protease Sensing. ACS Nano 6, 11044-11058(2012).
9. Hildebrandt, N., Spillmann, C. M., Algar, W. R., Pons, T., Stewart, M. H., Oh, E., Susumu, K., Díaz, S. A., Delehanty, J. B., Medintz, I. L. Energy Transfer with Semiconductor Quantum Dot Bioconjugates: A Versatile Platform for Biosensing, Energy Harvesting, and Other Developing Applications. Chemical Reviews 117, 536-711 (2017).
10. Diaz, S. A., Breger, J. C., Medintz, I. L. Monitoring Enzymatic Proteolysis Using Either Enzyme- or Substrate Bioconjugated Quantum Dots. Methods in Enzymology 571, 19-54 (2016).
11. Boeneman, K., Prasuhn, D., Blanco-Canosa, J., Dawson, P., Melinger, J. S., Ancona, M., Stewart, M., Susumu, K., Huston, A., Medintz, I. L. Self-assembled quantum dot-sensitized multivalent photonic wires. Journal of the American Chemical Society 132, 18177-18190 (2010).
12. Klein, W. P, Diaz, S. A., Chiriboga, M., Walper, S. A., Medintz, I. L. Dendimeric DNA-Based Nanoscaffolded BRET-FRET Optical Encryption Keys. ACS Applied Nano Materials. 2(12), 7459-7465 (2019).
13. Massey, M., Medintz, I., Ancona, M., Algar, R. Time-Gated FRET and DNA-Based Photonic Molecular Logic Gates: AND, OR, NAND, and NOR. ACS Sensors. 2(8), 1205-1214 (2017).
14. Claussen, J., Hildebrandt, N., Susumu, K., Ancona, M., Medintz, I. Complex Logic Functions Implemented with Quantum Dot Bionanophotonic Circuits. ACS Applied Materials and Interfaces. 6(6), 3771-3778 (2014).

What is claimed is:

1. A modular energy transfer platform comprising:
a CdSe/ZnS core-shell quantum dot (QD) scaffold having four additional components bound thereto, the additional components comprising terbium, ruthenium, AlexaFluor 647, and Cy5.5, wherein
each of the additional components are bound to the QD scaffold via an arm, each arm comprising a peptide-PNA strand comprising (1) a peptide nucleic acid (PNA) comprising a PNA sequence and (2) a peptide comprising a polyhistidine sequence effective to bind the peptide-PNA strand to the QD; and
the arm comprising ruthenium features the ruthenium conjugated to the PNA sequence via a modified peptide derivative.

2. The modular energy transfer platform of claim 1 wherein the arm comprising ruthenium features the ruthenium conjugated to the PNA sequence via a modified peptide derivative substituted for an arm comprising ruthenium featuring the ruthenium conjugated to a DNA sequence.

3. The modular energy transfer platform of claim 1 wherein the CdSe/ZnS core-shell quantum dot (QD) is substituted for a QD that emits at a wavelength and FRET dye acceptors and donors along with electron transfer acceptors selected based on desired results.

4. A method of preparing a modular energy transfer platform, the method comprising:
heating a mixture of anchor, template, and probe strands under conditions sufficient to denature DNA found within, and allowing them to assemble into arms, and then
without further purification, allowing these arms to bind to a quantum dot to form the modular energy platform of claim 1.

5. The method of preparing a modular energy transfer platform of claim 4 further comprising the steps of:
assembling components to the quantum dot;
controlling ratios of the components assembled to the quantum dot; and
controlling the relative rate of FRET or electron transfer.

6. A method of analysis comprising:
contacting a modular energy transfer platform of claim 1 with a sample comprising (1) nucleic acids operable to displace the terbium, AlexaFluor 647, and/or Cy5.5 from the scaffold; and/or (2) a protease operable to separate the ruthenium from the scaffold; and
measuring a change in the optical properties of the scaffold as a result of the contacting.

7. The method of analysis of claim 6 further comprising:
inserting a target molecule between the QD and the dye, Tb, or Ru; and
detecting a hydrolytic substance.

8. The method of analysis of claim 6 further comprising:
detecting a complex multistep biological process wherein a protease site on a peptide is phosphorylated or dephosphorylated for cleavage to happen, thus providing an indirect sensor for that activity or coupled activity.
